(19) United States
(12) Patent Application Publication
(10) Pub. No.: US 2014/0315209 A1
(43) Pub. Date: Oct. 23, 2014

(54) MOLECULAR ASSAY FOR THE AMPLIFICATION AND DETECTION OF KPC GENES RESPONSIBLE FOR HIGH-LEVEL RESISTANCE TO CARBAPENEM IN GRAM NEGATIVE BACTERIA

(71) Applicant: GENEOHM SCIENCES CANADA, INC., Sainte-Foy (CA)

(72) Inventors: Catherine Lippe, Sainte-Foy (CA); Isabelle Duteaud, Sainte-Foy (CA); Celine Roger Dalbert, Sainte-Foy (CA)

(21) Appl. No.: 14/362,085

(22) PCT Filed: Nov. 30, 2012
(86) PCT No.: PCT/CA2012/050868

§ 371 Date: May 30, 2014

Related U.S. Application Data
(60) Provisional application No. 61/565,620, filed on Dec. 1, 2011.

(51) Int. Cl. C12Q 1/68

(52) U.S. Cl. CPC C12Q 1/689
USPC 435/6.11; 435/6.12

(57) ABSTRACT

Methods and kits useful for the detection and identification of carbapenem-resistant pathogens harboring carbapenemase-encoding nucleic acids. Said methods comprise PCR amplification of a target region of the beta-lactamase encoding Klebsiella pneumoniae carbapenemase genes (bla_{KPC}) and variants thereof with a primer set comprising SEQ ID Nos: 1 and 2, and variants thereof

### REFERENCE TO SEQUENCE LISTING, TABLE, OR COMPUTER PROGRAM LISTING

The present application is being filed along with a sequence listing in electronic format. The sequence listing is provided as file entitled GENOM.102WO.txt, created Nov. 30, 2012 which is 13 KB in size. The information in the electronic format of the sequence listing is incorporated herein by reference in its entirety.

### BACKGROUND OF THE INVENTION

1. Field of the Invention

The embodiments disclosed herein relate to the field of molecular diagnostics, and in particular, diagnostic assays for detecting antibiotic resistant microorganisms.

2. Description of the Related Art

Antibiotic resistant pathogens have become an increasing problem in the clinical setting. β-lactams are a class of antibiotics that include penicillins, cephems, monobactams, and carbapenems. Carbapenems exhibit a broad spectrum of antibacterial activity and share a common backbone structure, shown in FIG. 1, which renders them highly resistant to β-lactamases. As such, there is a strong selective pressure for bacteria that have acquired resistance mechanisms to carbapenems. (Rasmussen et al., (1997) Antimicrob. Agents Chemother. 41:223-232).

Carbapenem resistance is known to arise from different mechanisms. In some cases, the carbapenem resistance arises from mutations in bacterial porin genes, which alter membrane permeability. In other cases, carbapenem resistance can arise from the acquisition of carbapenem mobile genetic elements that encode carbapenemase. Carbapenemases are carbapenem-hydrolyzing β-lactamases. The susceptibility patterns for isolates with a carbapenemase and those with porin mutations can be identical, thus rendering it impossible to differentiate using conventional antibiotic susceptibility assays.

As mentioned, one mechanism of carbapenem resistance is the production of a serine carbapenemase, KPC, also known as bla_{KPC}. KPC was originally detected and identified in a strain of Klebsiella pneumoniae in 2001. Yigit et al., (2001) Antimicrob. Ag. Chemother. 45(4):1151-1161 describes the cloning of and sequence of the KPC-1 gene from the K. pneumoniae strain 1534, isolated in a hospital setting in 1996. K. pneumoniae is the causative agent for various pathologies, such as intestinal infections, urinary tract infections, wound or surgical site infections, pneumonia and blood infections. K. pneumoniae is also implicated in hospital acquired infections (HAI).

Following the initial description of KPC-1 from K. pneumoniae in 2001, carbapenem resistance in Klebsiella has been rapidly increasing. KPC enzymes have become endemic in the Northeastern/Mid-Atlantic region of the United States, with surveillance cultures of hospitals in the New York City area reporting rates of carbapenem resistance in K. pneumoniae isolates ranging up to 24%. See, Woodford et al. (2004) Antimicrob. Agents Chemother. 48:4793-4799. Thus, detection and surveillance of carbapenemase-producing organisms have become matters of major importance for the selection of appropriate therapeutic schemes and the implementation of infection control measures. See, Yigit, et al. (2001) Antimicrob. Agents Chemother. 45:1151-1161. bla_{KPC}-producing bacteria are usually resistant to virtually all classes of antibiotics-β-lactam agents, including penicillins, cephalosporins, monobactams, and carbapenems—leaving physicians with limited antibiotic choices for treating infected patients.

In addition to the rapid spread of carbapenem resistance among strains of K. pneumoniae, since 1996, kpc genes have been isolated and identified in other, clinically relevant Enterobacteriaceae species including Klebsiella oxytoca, Salmonella enterica, Enterobacter cloacae, Escherichia coli, and Citrobacter freundii, as well as in Pseudomonas aeruginosa. Many of the kpc harboring microorganisms are commonly found in human and other animals' intestinal flora, as well as water or soil. See, Manual of Microbiology, 8^{th} edition, Ed. P. R. Murray, et al., ASM Press, Washington, D.C., 2003. The genes encoding the bla_{KPC} enzymes are usually flanked by transposon-related sequences that have been identified on transferable plasmids, thus giving them the potential to disseminate rapidly in the clinical settings.

In view of the foregoing, the detection and surveillance of carbapenemase-producing microorganisms has become critically important. Conventional, manual, antimicrobial susceptibility testing fails to provide an adequate solution to this need. Not only is the manual susceptibility testing time consuming (typically requiring between 48 to 96 hours), but it is plagued by inaccuracies that can arise from any number of circumstances, such as improper storage of antibiotic disk, improper diffusion of some antibiotic disks, and a lack of standardization of the process. To further compound the issue, the levels of enzymes produced by carbapenem resistant bacteria (harboring a carbapenemase gene) are often low. As such, routine susceptibility testing methods, which are based on the minimum inhibitory concentration of antibiotics, can fail to detect the presence of a KPC β-lactamase producing organism. Consequently, a KPC gene-carrying organism can be resistant to a carbapenem treatment, even though it demonstrates in vitro susceptibility when using the breakpoints suggested by the current CLSI (CLSI, M100-S 18). Finally, clinical laboratories can have problems differentiating carbapenemase-producing microorganisms from microoganisms having an extended-spectrum β-lactamase (ESBL), as the commonly-used confirmation tests are extremely similar. Specifically, both tests include clavulanate-potentiated activities of ceftriaxone, ceftazidime, cefepime, and aztreonam. A carbapenem-hydrolysing β-lactamase could be mistaken as an ESBL producer.

Yigit et al. (2001) supra, describes a Polymerase Chain Reaction (PCR) method for specific amplification of the KPC-1 gene using specific primers without probe, necessitating the sequencing of the PCR product (Yigit et al. 2001). This method allows the detection of the KPC gene in less time than the susceptibility testing, but it still takes extra time to sequence and analyze the results generated by the PCR. Furthermore, several different carbapenems have been identified since Yigit et al. was published, and the Yigit et al. method is not optimal for detection of the later-identified KPC genes.

International Patent Application Publication No. WO 2008/124670 discloses a nucleic acid amplification-based method for detection of KPC carbapenemase genes, which, in contrast to the Yigit et al. assay, used probes to enable more rapid detection of amplification products. In addition, the assay described in WO 2008/124670 enabled the detection of two additional KPC genes, bla_{KPC-2}, bla_{KPC-3}.

To date, at least eight additional bla_{KPC} genes have been described, i.e., bla_{KPC-4} through bla_{KPC-11}, which represent additional variants with additional nucleotide sequence differences. There is a continuing need for highly sensitive and specific diagnostic tools to detect and identify emerging carbapenemase-resistant pathogens, including those harboring newly-identified carbapenemase isoforms. Provided herein are improved compositions and methods for the detection and identification of carbapenem-resistant bacteria.

### SUMMARY OF THE INVENTION

Provided herein are methods and kits that can be used to detect known isoforms of KPC beta lactamases, and in particular that advantageously detect isoforms 1-11 of KPC beta-lactamases (bla_{KPC1-11}). The kits can include an amplification primer, or amplification primer pair. In some embodiments, the kits include at least a forward and reverse amplification primer, wherein the forward and reverse amplification primers are substantially complementary to, or fully complementary to, SEQ ID NOs: 19-28, or the complements thereof, over the entire primer sequence. The forward and reverse primers together are capable of amplifying a target amplicon from SEQ ID NOs: 19-28, e.g., under standard PCR conditions. Accordingly, in some embodiments, the forward primer and the reverse primer each comprise between 10 to 45 nucleotides. The forward primer can include at least 10 consecutive nucleotides of SEQ ID NO:1, and wherein the reverse primer can include at least 10 consecutive nucleotides of SEQ ID NO:2. In some embodiments, wherein said forward primer consists of SEQ ID NO:1, or a variant thereof, wherein said variant can include 1 to 5 nucleotide additions or deletions at its 5′ end, its 3′ end or both, and 1 to 5 degenerate bases, wherein said reverse primer consists of SEQ ID NO:2, or a variant thereof, wherein said variant can include 1 to 5 nucleotide additions or deletions at its 5′ end, its 3′ end or both, and 1 to 5 degenerate bases.

In some embodiments, the kits can also include a probe that comprises a nucleic acid sequence that is substantially complementary to at least a portion of the target amplicon. In some embodiments, the probe includes detectable moiety on its 3′ end. In some embodiments, the probe includes a detectable moiety on its 5′ end. The probe can be an oligonucleotide between 10 and 45 bases in length, wherein at least 15 consecutive bases of the oligonucleotide are substantially complementary a sequence within the target amplicon. In some embodiments, the probe includes an oligonucleotide between 10 and 45 bases in length, wherein said oligonucleotide comprises SEQ ID NO:3. For example in some embodiments, the probe comprises an oligonucleotide, wherein the oligonucleotide consists of SEQ ID NO:15. In some embodiments, the probe is a TaqMan® probe that comprises SEQ ID NO: 3. In some embodiments, the probe is a Molecular Beacon probe that comprises SEQ ID NO: 3.

The primers and probes of the kits disclosed herein can be dried, e.g., lyophilized. In some embodiments, the kits include reagents for nucleic acid amplification reactions. In some embodiments, for example, the kits can include dNTPs. In some embodiments, the kits can include a reaction buffer. In some embodiments, the kits can include a polymerase. In some embodiments, the kits can include any combination of reagents, e.g., any combination of buffers, enzymes, dNTPs, and the like.

In some embodiments, the kits can include a positive control nucleic acid. For example, some embodiments provide kits that include positive control nucleic acids that comprises a sequence substantially complementary to the forward primer and a sequence that is substantially complementary to the reverse primer, and wherein the remainder of the positive control nucleic acid is not substantially complementary to any one of SEQ ID NOs: 19-28, or the complements thereof.

Also provided herein are methods for determining the presence of a carbapenem-resistant pathogen in a sample, as well as methods for determining the presence of KPC sequences of isoforms 1-11 of KPC beta-lactamases (bla_{KPC1-11}). The methods can include the steps of providing the sample and contacting the sample with a forward amplification primer and a reverse amplification primer, wherein said forward and reverse amplification primers are substantially complementary or fully complementary to SEQ ID NOs: 19-29, or the complement thereof, through the length of the primers, and wherein the forward and reverse amplification primers are together capable of specifically amplifying a target amplicon from SEQ ID NOs: 19-29. The contacting step can occur under standard nucleic acid amplification conditions, e.g., PCR conditions, or the like, such that the target amplicon is generated provided that the sample comprises the carbapenem-resistant pathogen, or the KPC sequences of isoforms 1-11 of KPC beta-lactamases (bla_{KPC1-11}), to generate an amplified sample. The methods can also include determining whether the target amplicon is present in the amplified sample. The method of claim 15, wherein the generation of the amplified sample comprises real time PCR.

In some embodiments, the method of determining whether the target amplicon is present in the amplified sample can include the step of contacting the amplified sample with a probe, wherein the probe comprises a detectable moiety, and wherein said detectable moiety generates a signal in the presence of the target amplicon.

In some embodiments, the methods can include the step of providing a positive internal control nucleic acid that includes a sequence substantially complementary to the forward primer and a sequence that is substantially complementary to the reverse primer, and wherein the remainder of the positive control nucleic acid is not substantially complementary to any one of SEQ ID NOs: 19-29, or the complements thereof. In some embodiments, the method further includes contacting said positive control nucleic acid with the forward and said reverse amplification primers under the standard nucleic acid amplification conditions to generate a positive control amplicon. The positive control amplicon can be detected.

### BRIEF DESCRIPTION OF THE DRAWINGS

FIG. 1 shows the chemical structure of the common backbone of carbapenems.

FIGS. 2A-2B show agarose gels of amplification reactions as described herein.

FIG. 3 shows an alignment of SEQ ID NOs: 1-3 with bla_{KPC-1} through bla_{KPC-11}.

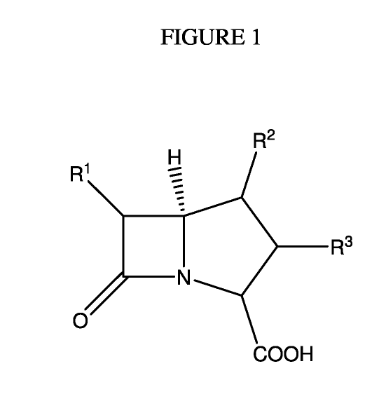

### DETAILED DESCRIPTION OF THE PREFERRED EMBODIMENT

Provided herein are improved, highly specific and highly sensitive compositions and methods of using the same, for the detection of carbapenemase genes, e.g., from clinical samples.

### Specimens and Samples

The embodiments disclosed herein can be used to detect and/or identify carbapenemase-harboring bacteria in a specimen. As used herein, the term “specimen” can refer to a clinical specimen or sample from one or any number of sources, including, but not limited to, bodily fluids (including, but not limited to, blood, urine, serum, lymph, saliva, anal and vaginal secretions, perspiration, peritoneal fluid, pleural fluid, effusions, ascites, purulent secretions, lavage fluids, drained fluids, brush cytology specimens, biopsy tissue, explanted medical devices, infected catheters, pus, biofilms and semen) of virtually any organism, with mammalian samples, particularly human samples, and environmental samples (including, but not limited to, air, agricultural, water and soil samples) finding use in the invention. In addition, samples can be taken from food processing, which can include both input samples (e.g. grains, milk or animal carcasses), samples in intermediate steps of processing, as well as finished food ready for the consumer. As most carbapenem resistant microbes are Enterobacteriaciae, the embodiments disclosed herein are particularly useful in the analysis of specimens and samples from blood, feces, urine, and nasal swabs, and are particularly useful for detection of carbapenem resistant Gram negative microbes.

The embodiments disclosed herein are advantageously adapted to provide highly specific detection of carbapenemase harboring pathogens is in blood (e.g. wound samples), urine, fecal samples, and nasal swabs. In some embodiments, samples suspected of containing a carbapenem-resistant pathogen can be analyzed directly. In a preferred embodiment, the samples are direct samples. A “direct sample” is a sample that is collected from a subject and screened using the methods disclosed herein without isolating or culturing bacteria from the sample. The direct samples are generally only minimally processed prior to screening. In various embodiments, the samples may be lysed using any acceptable method known in the art and centrifuged to remove cellular debris. The supernatant is retained for screening. In another embodiment, the nucleic acid is pelleted, washed, and resuspended in appropriate buffer prior to screening in the methods disclosed herein. In other words, the direct samples can be contacted with the requisite components to perform a nucleic acid amplification assay as disclosed herein, without the need to culture and with minimal sample manipulation.

### Primers and Probes

In some embodiments, the specimen or sample can be contacted with a set of amplification primers. In some embodiments, the specimen or sample can be contacted with a probe. As used herein, the terms “primer” and “probe” include, but are not limited to oligonucleotides or nucleic acids. The terms “primer” and “probe” encompass molecules that are analogs of nucleotides, as well as nucleotides. Nucleotides and polynucleotides, as used herein shall be generic to polydeoxyribonucleotides (containing 2-deoxy-D-ribose), to polyribonucleotides (containing D-ribose), to any other type of polynucleotide which is an N- or C-glycoside of a purine or pyrimidine base, and to other polymers containing nonnucleotidic backbones, for example, polyamide (e.g., peptide nucleic acids (PNAs)) and polymorpholino (commercially available from the Anti-Virals, Inc., Corvallis, Oreg., as NEUGENE™ polymers), and other synthetic sequence-specific nucleic acid polymers providing that the polymers contain nucleobases in a configuration which allows for base pairing and base stacking, such as is found in DNA and RNA.

The terms nucleotide and polynucleotide include, for example, 3′-deoxy-2′,5′-DNA, oligodeoxyribonucleotide N3′→P5′ phosphoramidates, 2′-O-alkyl-substituted RNA, double- and single-stranded DNA, as well as double- and single-stranded RNA, DNA:RNA hybrids, and hybrids between PNAs and DNA or RNA. The terms also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine. The resultant modified nucleotide or polynucleotide may form a base pair with other such modified nucleotidic units but not with A, T, C, G or U. For example, guanosine (2-amino-6-oxy-9-beta.-D-ribofuranosyl-purine) may be modified to form isoguanosine (2-oxy-6-amino-9-.beta.-D-ribofuranosyl-purine). Such modification results in a nucleoside base which will no longer effectively form a standard base pair with cytosine. However, modification of cytosine (1-.beta.-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al., supra, and Mantsch et al. (1993) Biochem. 14:5593-5601, or by the method described U.S. Pat. No. 5,780,610 to Collins et al. The non-natural base pairs referred to as κ and π, may be synthesized by the method described in Piccirilli et al. (1990) Nature 343:33-37 for the synthesis of 2,6-diaminopyrimidine and its complement (1-methylpyrazolo[4,3]-pyrimidine-5,7-(4H,6H)-dione. Other such modified nucleotidic units which form unique base pairs have been described in Leach et al. (1992) J. Am. Chem. Soc. 114:3675-3683 and Switzer et al., supra, or will be apparent to those of ordinary skill in the art.

Preferably, the set of amplification primers comprises at least one, two, three, or four, or more primers and/or probes that each contain one or more universal bases. As used herein, the term “universal base” refers to a nucleotide analog that can hybridize to more than one nucleotide selected from A, T, C, and G. In some embodiments, the universal base can be selected from the group consisting of deoxyinosine, 3-ntiropyrrole, 4-nitroindole, 6-nitroindole, 5-nitroindole. Preferably, the universal base is deoxyinosine. In some embodiments, the set of amplification primers, and probes disclosed herein include at least one primer and/or probe that has one, two, three, four, five, six, seven, eight, nine, ten, or more universal bases.

Oligonucleotide primers and/or probes can preferably be between 10 and 45 nucleotides in length. For example, the primers and or probes can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers and/or probes can be provided in any suitable form, included bound to a solid support, liquid, and lyophilized, for example. The primer and probe sequences disclosed herein can be modified to contain additional nucleotides at the 5′ or the 3′ terminus. The skilled artisan will appreciate, however, that additional bases to the 3′ terminus of amplification primers (not necessarily probes) must be complementary to the target sequence.

The primer and probe sequences may be modified by having nucleotide substitutions (relative to the target sequence) within the oligonucleotide sequence, provided that the oligonucleotide contains enough complementarity to hybridize specifically to the target nucleic acid sequence. In this manner, at least 1, 2, 3, 4, or up to about 5 nucleotides can be substituted. As used herein, the term “complementary” refers to sequence complementarity between regions of two polynucleotide strands or between two regions of the same polynucleotide strand. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Fully complementary” refers to a first polynucleotide that is 100% or “fully” complementary to a second polynucleotide and thus forms a base pair at every nucleotide position. “Partially complementary” also refers to a first polynucleotide that is not 100% complementary (e.g., 90%, or 80% or 70% complementary) and contains mismatched nucleotides at one or more nucleotide positions.

In some embodiments, the oligonucleotides disclosed herein are fully or substantially complementary to a target sequence or target polynucleotide. As used herein, the terms “target polynucleotide” and “target nucleic acid” refer to a polynucleotide whose presence is to be determined in a sample. In the embodiments disclosed herein, the target nucleic acid corresponds to nucleic acids that encode any of the carbapenemases. TABLE 1 below provides information regarding the sequences of various isoforms of bla_{KPC} known to date, including bla_{KPC-1} through bla_{KPC-11}. In preferred embodiments, the primer and probes set forth herein are capable of specific amplification and detection of various carbapenemase sequences in any enterobacteria and/or Pseudomonad, or any other microorganism found to harbor any of the bla_{KPC} isoforms as described herein.

TABLE 1
KPC ISOFORMS

| | Nucleo- | | GENBANK ® | |
|---|---|---|---|---|
| | tide Po- | Base | Accession | |
| Enzyme | sition | Change | No | Species |
| KPC-1 | 520 | G → A | AF297554 | Klebsiella pneumoniae |
| KPC-2** | | | AY034847 | Klebsiella pneumoniae |
| | | | AF481906 | Salmonella enterica |
| | | | | subsp. |
| | | | AY210886 | Klebsiella oxytoca |
| | | | DQ989640 | Enterobacter cloacae |
| | | | DQ989639 | Escherichia coli |
| | | | DQ523564 | Klebsiella pneumoniae |
| | | | EF062508 | Citrobacter freundii |
| | | | EU244644 | Klebsiella pneumoniae |
| | | | EU784136 | Klebsiella pneumoniae |
| | | | DQ897687 | Klebsiella pneumoniae |
| KPC-3 | 814 | C → T | AF395881 | Klebsiella pneumoniae |
| | | | AM774409 | Enterobacter cloacae |
| KPC-4 | 308 | C → G | AY700571 | Enterobacter sp. |
| | 716 | T → G | EU447304 | Klebsiella pneumoniae |
| KPC-5 | 308 | C → G | EU400222 | Pseudomonas aeruginosa |
| KPC-6 | 716 | T → G | EU555534 | Klebsiella pneumoniae |
| KPC-7 | 147 | G → A | EU729727 | Klebsiella pneumoniae |
| | 814 | C → T | | |
| KPC-8 | 716 | T → G | FJ234412 | Klebsiella pneumoniae |
| | 814 | C → T | | |
| KPC-9 | 716 | T → C | FJ624872 | Escherichia coli |
| | 814 | C → T | | |
| KPC-10 | 308 | C → G | GQ140348 | Acinetobacter baumanii |
| | 814 | C → T | | |
| KPC-11 | 308 | C → T | HM066995.1 | Klebsiella pneumoniae |

**The KPC-2 gene does not contain any of the mutations mentioned above.

In preferred embodiments, the amplification primers disclosed herein are 100% complementary to all of the KPC isoforms disclosed herein. This is in contrast to other molecular assays for the detection of carbapenemase genes, e.g., as described in Yigit et al., supra, and in International Patent Application Publication No. WO 08/124670. In fact, at least one primer in each of the assays described in WO 08/124670 harbors a mismatch, when compared the sequences of bla_{KPC-9}, and/or bla_{KPC-10}, and/or bla_{KPC-11}.

As used herein, the term “hybridization” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tₘ) of the formed hybrid, the presence of other components (e.g., the presence or absence of polyethylene glycol), the molarity of the hybridizing strands and the G:C content of the polynucleotide strands. In one embodiment, the primers are designed such that the Tₘ of one primer in the set is within 2° C. of the Tₘ of the other primer in the set. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al, eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). As discussed further herein, the term “specific hybridization” or “specifically hybridizes” refers to the hybridization of a polynucleotide, e.g., an oligonucleotide primer or probe or the like to a target sequence, such as a bla_{KPC} target sequence, a positive control target nucleic acid sequence, or the like, and not to unrelated sequences, under conditions typically used for nucleic acid amplification.

The primers described herein can be prepared using techniques known in the art, including, but not limited to, cloning and digestion of the appropriate sequences and direct chemical synthesis. Chemical synthesis methods that can be used to make the primers of the described herein, include, but are not limited to, the phosphotriester method described by Narang et al. (1979) Methods in Enzymology 68:90, the phosphodiester method disclosed by Brown et al. (1979) Methods in Enzymology 68:109, the diethylphosphoramidate method disclosed by Beaucage et al. (1981) Tetrahedron Letters 22:1859, and the solid support method described in U.S. Pat. No. 4,458,066. The use of an automated oligonucleotide synthesizer to prepare synthetic oligonucleotide primers described herein is also contemplated herein. Additionally, if desired, the primers can be labeled using techniques known in the art and described below.

In some embodiments, the primers and/or probes include oligonucleotides that hybridize to a target nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “substantially complementary” refers to the complementarity between two nucleic acids, e.g., the complementary region of the oligonucleotide and the target sequence. The complementarity need not be perfect; there may be any number of base pair mismatches that between the two nucleic acids. However, if the number of mismatches is so great that no hybridization can occur under even the least stringent of hybridization conditions, the sequence is not a substantially complementary sequence. When two sequences are referred to as “substantially complementary” herein, it is meant that the sequences are sufficiently complementary to the each other to hybridize under the selected reaction conditions. The relationship of nucleic acid complementarity and stringency of hybridization sufficient to achieve specificity is well known in the art and described further below in reference to sequence identity, melting temperature and hybridization conditions. Therefore, substantially complementary sequences can be used in any of the detection methods of the invention. Such probes can be, for example, perfectly complementary or can contain from 1 to many mismatches so long as the hybridization conditions are sufficient to allow, for example discrimination between a target sequence and a non-target sequence. Accordingly, substantially complementary sequences can refer to sequences ranging in percent identity from 100, 99, 98, 97, 96, 95, 94, 93, 92, 91, 90, 89, 85, 80, 75 or less, or any number in between, compared to the reference sequence. For example, the oligonucleotides disclosed herein can contain 1, 2, 3, 4, 5, or more mismatches and/or degenerate bases, as compared to the target sequence to which the oligonucleotide hybridizes, with the proviso that the oligonucleotides are capable of specifically hybridizing to the target sequence under, for example, standard nucleic acid amplification conditions.

### Primer Pairs

In some embodiments, the set of amplification primers includes one or more, e.g., 1, 2, 3, 4, 5, 6, 7, 8, 9, 10, 11, 12, 13, 14, 15, or more primer pairs. As used herein, the term “primer pair” can refer to two primers that individually hybridize to opposite strands of a target nucleic acid, e.g., a KPC-encoding nucleic acid or gene or fragment thereof, or the like, wherein each primer can be extended at its 3′ end to form a target amplification product, for example in a polymerase chain reaction (PCR). Primer pairs can include forward and reverse primers. Preferably, the compositions, methods and kits disclosed herein include one KPC-specific primer pair. In some embodiments, the KPC-specific primer pair, in addition to being specific for bla_{KPC} nucleic acids, is specific for a positive control sequence (e.g., a recombinant nucleic acid that is unrelated to bla_{KPC} nucleic acids, yet is engineered to include sequences complementary to the KPC-specific primer pair), as discussed in further detail below.

In some embodiments, the compositions and methods disclosed herein include a primer pair that comprises at least one set of amplification primers that hybridize to a bla_{KPC} gene. For example, the compositions and methods disclosed herein can be used to detect and/or identify bla_{KPC} beta-lactamases from a bacterium listed in Table 1. In some embodiments, the compositions and methods include a plurality of amplification primers, which collectively enable the detection and identification bla_{KPC} carbapenemases from all of the bacteria listed in Table 1. In some embodiments, a single primer pair can be used for the detection and identification of all of the various bla_{KPC} carbapenemase isoforms from all of the bacteria listed in Table 1. In some embodiments, the compositions and method disclosed herein include primer pairs (or a single primer pair) that collectively hybridize to and amplify nucleic acids of at least two (e.g., all eleven) bla_{KPC} isoforms selected from bla_{KPC}-1, bla_{KPC}-2, bla_{KPC}-3, bla_{KPC}-4, bla_{KPC}-5, bla_{KPC}-6, bla_{KPC}-7, bla_{KPC}-8, bla_{KPC}-9, bla_{KPC}-10, and bla_{KPC}-11. Primers useful for the detection and identification of various isolates of bla_{KPC} include, for example, oligonucleotides that have at least 10 consecutive nucleic acids of SEQ ID NOs: 1-14 or the complements thereof.

The bla_{KPC} primers disclosed herein advantageously do not have any mismatches, and are 100% complementary to, bla_{KPC-11}. Yigit et al., supra, describes a PCR amplification reaction using primers that are not 100% complementary to all bla_{KPC} isoforms. WO 08/124670 describes 7 different primer and probe combinations for the amplification and detection of carbapenem-resistant pathogens. In contrast to the present assay, none of the 7 different primer and probe combinations are 100% complementary to all of the presently known isoforms of bla_{KPC}, including isoforms 1-11. Accordingly, the primers and probes of the present embodiments exhibit improved specificity and sensitivity for detection of carbapenem-resistant pathogens.

In some embodiments, primers can be used in pairs, e.g., in a PCR assay. For example, in some embodiments, the following forward and reverse primers are used together in an amplification assay: SEQ ID NOs: 1 and 2; SEQ ID NOs: 1 and 13, SEQ ID NOs: 1 and 14, SEQ ID NOs: 10 and 2, SEQ ID NOs: 10 and 13, SEQ ID NOs: 10 and 14; SEQ ID NOs: 4 and 5, and SEQ ID NOs: 7 and 8. In some embodiments, more than one primer pair can be used in an assay as described herein. For example, in some embodiments, 2, 3, 4, or more, primer pairs disclosed herein can be used together.

In some embodiments, variants of the primers and probes disclosed herein can be used in the assays described herein, with the proviso that the amplification primers retain their capability to specifically amplify target sequences, and with the proviso that oligonucleotide probes retain their capability to specifically hybridize to their target sequences. By way of example only, variants of the primers and/or probes useful in the embodiments disclosed herein can include additional bases on the 5′ or 3′ end. By way of example, variants of SEQ ID NO:1 that include additional bases can include additional bases on the 3′ end. If the additional bases are added to the 3′ end of SEQ ID NO:1, the bases should be 100% complementary to the target KPC sequences. The skilled artisan will appreciate that in the context of adding bases onto the 5′ end of an amplification primer, it is not critical that the additional bases are 100% complementary to the target KPC sequences, as the primer can still be extended from its 3′ end. For example, in some embodiments, amplification primers and/or probes can include up to 1, 2, 3, 4, 5, 6, 7, 8, 9, 10, 11, 12, 13, 14, 15, 16, 17, 18, 19, 20, or more, additional bases on the 3′ or 5′ end.

In addition to variants that include additional bases at either the 5′ or 3′ end, some embodiments provide primer and/or oligonucleotide probes that are shorter that the primers and/or probes described herein. For example, in some embodiments, the primers and/or probes can be 1, 2, 3, 4, 5 6, 7, 8, 9, 10, 11, 12, 13, 14, 15, 16, 17, 18, 19, or 20 nucleotides shorter than the sequences of SEQ ID NOs: 1-17, with the proviso that the primers and/or probes still retain their ability to specifically hybridize to their cognate target sequences. Further, primer variants that are shorter must still retain their ability to function as amplification primers in the methods disclosed herein. The skilled artisan will also readily appreciate that the primers and/or probes can be longer on the 3′ end and shorter on the 5′ end, or vice versa.

Still other variants of the primers and probes disclosed herein include primers and probes that have base mismatches, or include degenerate bases, as discussed elsewhere herein.

In some embodiments, amplification primers of an amplification primer pair have Tₘ's that are less than 10° C., less than 9° C., less than 8° C., less than 7° C., less than 6° C., less than 5° C., less than 4° C., less than 3° C., less than 2° C., or less than 1° C., apart from each other. In preferred embodiments, the primer pairs disclosed herein comprise a first and a second primer, wherein the different in Tₘ between the first and second primer is less than about 3° C.

As used herein, the term “Tₘ” and “melting temperature” are interchangeable terms which refer to the temperature at which 50% of a population of double stranded polynucleotide molecules become dissociated into single strands. The Tm of particular nucleic acids, e.g., primers, or oligonucleotide probes, or the like can be readily calculated by the following equation: Tₘ=69.3+0.41×(G+C)%−650/L, wherein L refers to the length of the nucleic acid. The Tₘ of a hybrid polynucleotide may also be estimated using a formula adopted from hybridization assays in 1 M salt, and is commonly used for calculating the Tₘ for PCR primers: [(number of A+T)×2° C.+(number of G+C)×4° C.], see, for example, Newton et al. (1997) PCR (2nd ed; Springer-Verlag, New York). Other more sophisticated computations exist in the art, which take structural as well as sequence characteristics into account for the calculation of Tₘ. A calculated Tₘ is merely an estimate; the optimum temperature is commonly determined empirically.

In some embodiments, binding or annealing of the primers and/or probes to target nucleic acid sequences is accomplished through hybridization. It will be appreciated by one skilled in the art that specific hybridization is achieved by selecting sequences which are at least substantially complementary to the target or reference nucleic acid sequence. This includes base-pairing of the oligonucleotide target nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below.

In some embodiments, the sample or specimen is contacted with a set of amplification primers and a probe. Preferably, the amplification primers and probes hybridize to target nucleic acids under a single set of conditions, i.e., stringent conditions, including standard PCR conditions discussed below. Stringent hybridization conditions can vary (for example from salt concentrations of less than about 1 M, more usually less than about 500 mM and preferably less than about 200 mM) and hybridization temperatures can range (for example, from as low as 0° C. to greater than 22° C., greater than about 30° C. and (most often) in excess of about 37° C. depending upon the lengths and/or the nucleic acid composition of the probes. Longer fragments may require higher hybridization temperatures for specific hybridization. As several factors affect the stringency of hybridization, the combination of parameters is more important than the absolute measure of a single factor. Accordingly, by way of example, the term “stringent hybridization conditions” can refer to either or both of the following: a) 6×SSC at about 45° C., followed by one or more washes in 0.2×SSC, 0.1% SDS at 65° C., and b) 400 mM NaCl, 40 mM PIPES pH 6.4, 1 mM EDTA, 50° C. or 70° C. for 12-16 hours, followed by washing. In some embodiments, the term “stringent conditions” can refer to standard PCR conditions.

In some embodiments, the sample or specimen is contacted with a set of amplification primers under standard PCR conditions, which are discussed in further detail below. For a review of PCR technology, including standard PCR conditions, applied to clinical microbiology, see DNA Methods in Clinical Microbiology, Singleton P., published by Dordrecht; Boston: Kluwer Academic, (2000) Molecular Cloning to Genetic Engineering White, B. A. Ed. in Methods in Molecular Biology 67: Humana Press, Totowa (1997) and “PCR Methods and Applications”, from 1991 to 1995 (Cold Spring Harbor Laboratory Press). Non-limiting examples of “PCR conditions” include the conditions disclosed in the references cited herein, such as, for example, 50 mM KCl, 10 mM Tris-HCl (pH 9.0), 0.1% Triton X-100, 2.5 mM MgCl₂, with an annealing temperature of 72° C.; or 4 mM MgCl₂, 100 mM Tris, pH 8.3, 10 mM KCl, 5 mM (NH₄)₂SO₄, 0.15 mg BSA, 4% Trehalose, with an annealing temperature of 59° C., or 50 mM KCl, 10 mM Tris-HCl (pH 9.0), 0.1% Triton X-100, 2.5 mM MgCl₂, with an annealing temperature of 55° C., or the like.

### Probes

The compositions and methods disclosed herein can include one or more probes. For example, in some embodiments, a labeled probe can be used to detect the extension product, or amplicon, generated from the amplification of target (and, optionally, internal control) nucleic acids as described elsewhere herein. Any probe format utilizing a labeled probe comprising the sequences of the invention may be used, e.g., molecular beacon probes, SCORPION™ probes, sunrise probes, FRET probes, TAQMAN® probes, or the like as is known in the art or described elsewhere herein. In preferred embodiments, the probes are molecular beacon probes. In some embodiments, more than one probe is used in the detection and identification of a target and/or internal control amplicon.

In some embodiments, the probes comprise an oligonucleotide sequence and a detectable moiety. In some embodiments, the probes do not comprise an oligonucleotide sequence, as discussed below. In some embodiments, the probe can include a detectable label. Labels of interest include directly detectable and indirectly detectable radioactive or non-radioactive labels such as fluorescent dyes. Directly detectable labels refer to detectable moieties that provide a directly detectable signal without interaction with one or more additional chemical agents. Examples of directly detectable labels include fluorescent labels. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase. In many preferred embodiments, the label is a directly detectable label. Directly detectable labels of particular interest include fluorescent labels. Fluorescent labels that find use in the subject invention include a fluorophore moiety. Specific fluorescent dyes of interest include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like.

In preferred embodiments, the compositions and methods disclosed herein include a molecular beacon probe, a TAQMAN™ probe, or a SCORPION™ probe. For example, in some embodiments, the compositions and methods disclosed herein include one or more molecular beacon probes, wherein the probes comprise at least 10 consecutive nucleotides of SEQ ID NOs: 3, 6, or 9. In some embodiments, more than one probe, e.g., more than one molecular beacon, can be used in a single amplification reaction. For example, a first molecular beacon can be designed to have an oligonucleotide sequence complementary to a carbapenem sequence, e.g., a carbapenem amplicon generated using the methods disclosed herein. A second molecular beacon can be designed to include an oligonucleotide sequence complementary to an unrelated, positive control sequence, as discussed elsewhere herein. With the use of more than one molecular beacon probe in a single reaction, each fluorescent label of the molecular beacon is chosen to have a non-overlapping emission wavelength with other fluorescent label(s).

In some embodiments, the probe can be a double stranded DNA binding moiety, such as ethidium bromide, SYBER green, LC green, SYTO9, EVAGREEN® fluorescent dye, CHROMOFY®, BEBO, and the like, that fluoresces and produces a detectable signal in the presence of double stranded DNA.

Preferably, the embodiments disclosed herein use sequence specific probes, such as molecular beacon probes. Molecular beacon probes comprise four parts, namely a loop, a stem, a 5′ flourophore and a 3′ quencher dye. The loop comprises an oligonucleotide segment that is complementary or substantially complementary to a target and/or control amplicons, as described elsewhere herein. The stem refers to sequences flanking the loop that are located at the 5′ and 3′ sides of the loop and that are not substantially complementary to the target and/or control amplicon sequence. The 5′ and 3′ flanking sequences of the stem are complementary or substantially complementary to each other. In some embodiments, for example, the stem can include 3, 4, 5, 6, 7, 8, 9, 10, or more nucleotides on each of the 5′ end and 3′ end of the loop (segment that is substantially complementary to a target and/or control amplicon), that are not complementary or substantially complementary to the target amplicon or control amplicon. For example, molecular beacons derived from SEQ ID NO:3, can include flanking sequences as shown in SEQ ID NO:15; molecular beacons derived from SEQ ID NO: 6 can include flanking sequences as shown in SEQ ID NO:16; molecular beacons derived from SEQ ID NO:9 can include flanking sequences as shown in SEQ ID NO:17. The molecular beacons disclosed herein include 5′ fluorophores and 3′ quenchers, that are coupled to the 5′ and 3′ ends of the flanking sequences of the probe. Flourophore/quencher pairs useful in the compositions and methods disclosed herein are well-known in the art, and can be found, e.g., described in S. Maims, “Selection of Fluorophore and Quencher Pairs for Fluorescent Nucleic Acid Hybridization Probes” available at the world wide web site molecular-beacons.org/download/marras,mmb06%28335%293.pdf. Preferred molecular probes useful in the embodiments disclosed herein can include, e.g., a 5′ TET moiety paired with a 3′ Dabcyl moiety on the same molecular beacon, or, alternatively a 5′ FAM moiety paired with a 3′ Dabcl moiety on the same molecular beacon.

In some embodiments, the oligonucleotide probes disclosed herein have a Tₘ that is higher than the Tₘ of the primers of an amplification primer pair used in the methods disclosed herein. For example, in some embodiments, the probes, e.g., molecular beacon probes or the like, have a Tₘ that is greater than 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C., or more than either amplification primer used to generate an amplicon to which the oligonucleotide probe hybridizes. For example, a molecular beacon probe can have a Tₘ that is at least 5-10° C. higher than either amplification primer pair used to generate the amplicon to which the molecular beacon hybridizes.

In some embodiments, the following molecular beacons can be used together with the following amplification primer pairs:

| | Molecular Beacon |
|---|---|
| Amplification Primer Pair | (sequence including stem sequences) |
| SEQ ID NOs: 1 and 2 | SEQ ID NO: 3 (SEQ ID NO: 15) |
| SEQ ID NOs: 2 and 10 | SEQ ID NO: 3 (SEQ ID NO: 15) |
| SEQ ID NOs: 2 and 11 | SEQ ID NO: 3 (SEQ ID NO: 15) |
| SEQ ID NOs: 2 and 12 | SEQ ID NO: 3 (SEQ ID NO: 15) |
| SEQ ID NOs: 1 and 13 | SEQ ID NO: 3 (SEQ ID NO: 15) |
| SEQ ID NOs: 1 and 14 | SEQ ID NO: 3 (SEQ ID NO: 15) |
| SEQ ID NOs: 4 and 5 | SEQ ID NO: 6 (SEQ ID NO: 16) |
| SEQ ID NOs: 7 and 8 | SEQ ID NO: 9 (SEQ ID NO: 17) |

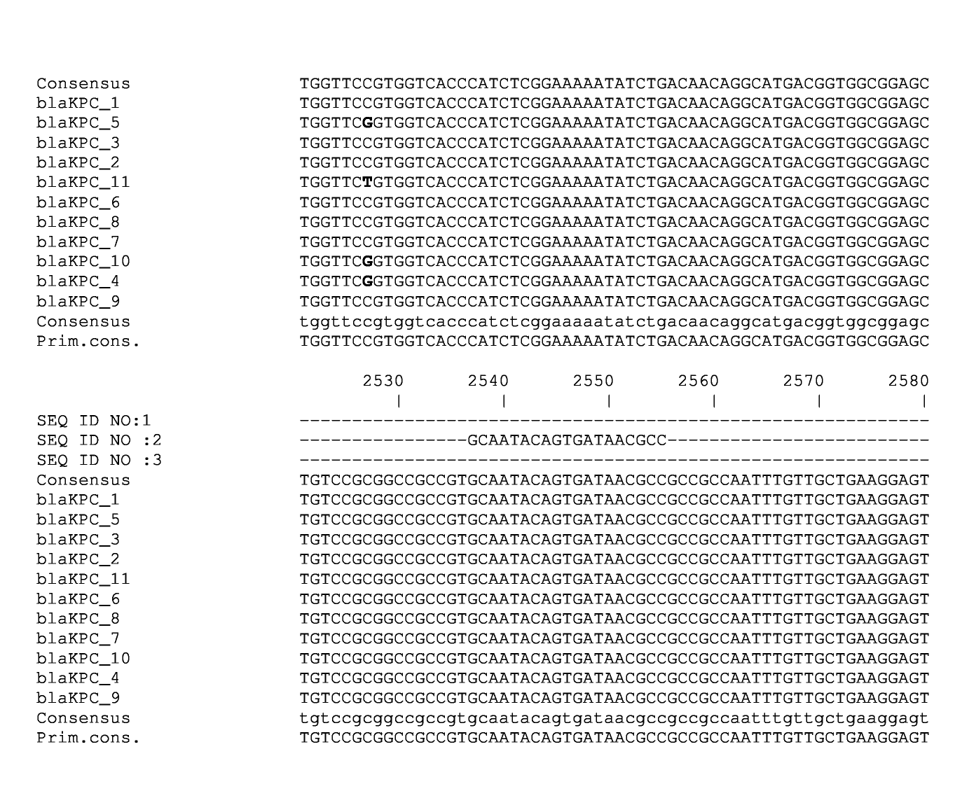

FIG. 3 shows an alignment of SEQ ID NOs: 1, 2 and 3 disclosed herein compared to sequences from each of the known 11 isoforms of bla_{KPC}. As shown, SEQ ID NOs: 1-3 are fully complementary to all 11 bla_{KPC} isoforms. The full complementarity of the sequences to all known isoforms of bla_{KPC} maximizes the specificity of the assay, thereby rendering the assays disclosed herein superior to other assays.

### Amplification

Some of the embodiments provided herein involve the specific amplification of KPC nucleic acids from samples. Accordingly, provided herein are methods for the specific amplification of KPC carbapenemase-encoding nucleic acids. Several methods for the specific amplification of target nucleic acids are known in the art, and are useful in the embodiments disclosed herein. Non-limiting examples of amplification methods include Polymerase Chain Reaction (PCR; see Saiki et al., 1985, Science 230:1350-1354, herein incorporated by reference), Ligase Chain Reaction (LCR; see Wu et al., 1989, Genomics 4:560-569; Barringer et al., 1990, Gene 89:117-122; Barany, 1991, Proc. Natl. Acad. Sci. USA 88:189-193, all of which are incorporated herein by reference), in situ hybridization, Transcription Mediated Amplification (TMA; see Kwoh et al., 1989, Proc. Natl. Acad. Sci. USA 86:1173-1177, herein incorporated by reference), Self-Sustaining Sequence Replication (3SR; see Guatelli et al., 1990, Proc. Natl. Acad. Sci. USA 87:1874-1878, herein incorporated by reference), Rolling Circle Amplification (RCA), Nucleic Acid Sequence Based Amplification (NASBA), Q 13 replicase system (Lizardi et al., 1988, BioTechnology 6:1197-1202, herein incorporated by reference) and Strand Displacement Amplification (SDA; see Walker et al., 1992, Proc. Natl. Acad. Sci. USA 89:392-396; Walker et al., 1992, Nuc. Acids. Res. 20:1691-1696; and EP 0 497 272, all of which are incorporated herein by reference)) including thermophilic SDA (tSDA).

In various embodiments, the methods disclosed herein are useful for detecting the presence of bla_{KPC} nucleic acids in samples having concentration of bacteria that is within physiological ranges (i.e., the concentration of bacteria in a sample collected from a subject infected with the bacteria). Thus, a sample can be directly screened without the need for isolating, concentrating, or expanding (e.g., culturing) the bacterial population in order to detect the presence of bla_{KPC} nucleic acids. In various embodiments, the methods disclosed herein are capable of detecting the presence of a carbapenem resistant pathogens from a sample that has a concentration of bacteria of about 1 CFU/ml, 10 CFU/ml, 100 CFU/ml, 1×10³ CFU/ml, 1×10³ CFU/ml, about 1×10⁴ CFU/ml, about 1×10⁵ CFU/ml, or about 1×10⁶ CFU/ml, or any number in between. As discussed in further detail below, the compositions and methods disclosed herein are more sensitive than known assays for carbapenem resistant pathogens, and can advantageously be used to detect carbapenemase nucleic acids of any known isoforms to date in a sample.

In some embodiments, the methods described herein provide for the detection and identification of pathogens harboring carbapenemase genes as disclosed herein, in real time, e.g., using the primers and probes disclosed herein in a PCR or QPCR assay. Numerous different PCR or QPCR protocols are known in the art and exemplified herein below and can be directly applied or adapted for use using the presently described compositions for the detection carbapenem resistant microorganisms in a sample.

Generally, in PCR, a target polynucleotide sequence is amplified by reaction with at least one oligonucleotide primer or pair of oligonucleotide primers. The primer(s) specifically hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence that is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, the BD MAX® (Becton Dickinson and Co., Franklin Lakes, N.J.), the VIPER® (Becton Dickinson and Co., Franklin Lakes, N.J.), the VIPER LT® (Becton Dickinson and Co., Franklin Lakes, N.J.), SMARTCYCLER® (Cepheid, Sunnyvale, Calif.), ABI PRISM 7700® (Applied Biosystems, Foster City, Calif.), ROTOR-GENE™; (Corbett Research, Sydney, Australia), LIGHTCYCLER® (Roche Diagnostics Corp, Indianapolis, Ind.), ICYCLER® (BioRad Laboratories, Hercules, Calif.) and MX4000® (Stratagene, La Jolla, Calif.).

Some embodiments provide methods including Quantitative PCR (QPCR) (also referred as real-time PCR). QPCR can provide quantitative measurements, and also provide the benefits of reduced time and contamination. As used herein, “quantitative PCR” (or “real time QPCR”) refers to the direct monitoring of the progress of a PCR amplification as it is occurring without the need for repeated sampling of the reaction products. In QPCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence (herein referred to as cycle threshold or “CT”) varies directly with the concentration of amplifiable targets at the beginning of the PCR process, enabling a measure of signal intensity to provide a measure of the amount of target nucleic acid in a sample in real time.

Methods for setting up PCR and QPCR are well known to those skilled in the art. The reaction mixture minimally comprises template nucleic acid (except in the case of a negative control as described below) and oligonucleotide primers and/or probes in combination with suitable buffers, salts, and the like, and an appropriate concentration of a nucleic acid polymerase. As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template until synthesis terminates. An appropriate concentration includes one that catalyzes this reaction in the presently described methods. Known DNA polymerases useful in the methods disclosed herein include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcusfuriosus (Pfu) DNA polymerase.

In addition to the above components, the reaction mixture of the present methods includes primers, probes, and deoxyribonucleoside triphosphates (dNTPs).

Usually the reaction mixture will further comprise four different types of dNTPs corresponding to the four naturally occurring nucleoside bases, i.e., dATP, dTTP, dCTP, and dGTP. In the methods of the invention, each dNTP will typically be present in an amount ranging from about 10 to 5000 μM, usually from about 20 to 1000 μM, about 100 to 800 μM, or about 300 to 600 μM.

The reaction mixture prepared in the first step of the methods of the invention further includes an aqueous buffer medium that includes a source of monovalent ions, a source of divalent cations, and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc, and the like, where the cation will typically be magnesium. Any convenient source of magnesium cation may be employed, including magnesium chloride, magnesium acetate, and the like. The amount of magnesium present in the buffer may range from 0.5 to 10 mM, and can range from about 1 to about 6 mM, or about 3 to about 5 mM. Representative buffering agents or salts that may be present in the buffer include Tris, Tricine, HEPES, MOPS, and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, for example, about pH 6.0, 6.5, 7.0, 7.5, 8.0, 8.5, 9.0, or 9.5. Other agents that may be present in the buffer medium include chelating agents, such as EDTA, EGTA, and the like. In some embodiments, the reaction mixture can include BSA, or the like. In addition, in some embodiments, the reactions can include a cryoprotectant, such as trehalose, particularly when the reagents are provided as a master mix, that can be stored over time.

In preparing the reaction mixture, the various constituent components may be combined in any convenient order. For example, the buffer may be combined with primer, polymerase, and then template nucleic acid, or all of the various constituent components may be combined at the same time to produce the reaction mixture.

Alternatively, commercially available premixed reagents can be utilized in the methods of the invention according to the manufacturer's instructions, or modified to improve reaction conditions (e.g., modification of buffer concentration, cation concentration, or dNTP concentration, as necessary), including, for example, TAQMAN® Universal PCR Master Mix (Applied Biosystems), OMNIMIX® or SMARTMIX® (Cepheid), IQ&#8482; Supermix (Bio-Rad Laboratories), LIGHTCYCLER® FastStart (Roche Applied Science, Indianapolis, Ind.), or BRILLIANT® QPCR Master Mix (Stratagene, La Jolla, Calif.).

Following preparation of the reaction mixture, the reaction mixture can be subjected to primer extension reaction conditions (“conditions sufficient to provide polymerase-based nucleic acid amplification products”), i.e., conditions that permit for polymerase-mediated primer extension by addition of nucleotides to the end of the primer molecule using the template strand as a template. In many embodiments, the primer extension reaction conditions are amplification conditions, which conditions include a plurality of reaction cycles, where each reaction cycle comprises: (1) a denaturation step, (2) an annealing step, and (3) a polymerization step. As discussed below, in some embodiments, the amplification protocol does not include a specific time dedicated to annealing, and instead comprises only specific times dedicated to denaturation and extension. The number of reaction cycles will vary depending on the application being performed, but will usually be at least 15, more usually at least 20, and may be as high as 60 or higher, where the number of different cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture will usually be raised to, and maintained at, a temperature ranging from about 85 to 100° C., usually from about 90 to 98° C., and more usually from about 93 to 96° C., for a period of time ranging from about 3 to 120 sec, usually from about 3 sec.

Following denaturation, the reaction mixture will be subjected to conditions sufficient for primer annealing to template nucleic acid present in the mixture (if present), and for polymerization of nucleotides to the primer ends in a manner such that the primer is extended in a 5′ to 3′ direction using the nucleic acid to which it is hybridized as a template, i.e., conditions sufficient for enzymatic production of primer extension product. In some embodiments, the annealing and extension processes occur in the same step. The temperature to which the reaction mixture is lowered to achieve these conditions will usually be chosen to provide optimal efficiency and specificity, and will generally range from about 50 to 75° C., usually from about 55 to 70° C., and more usually from about 60 to 68° C., more particularly around 60° C. Annealing conditions will be maintained for a period of time ranging from about 15 sec to 30 min, usually from about 20 sec to 5 min, or about 30 sec to 1 minute, or about 30 seconds.

This step can optionally comprise one of each of an annealing step and an extension step with variation and optimization of the temperature and length of time for each step. In a two-step annealing and extension, the annealing step is allowed to proceed as above. Following annealing of primer to template nucleic acid, the reaction mixture will be further subjected to conditions sufficient to provide for polymerization of nucleotides to the primer ends as above. To achieve polymerization conditions, the temperature of the reaction mixture will typically be raised to or maintained at a temperature ranging from about 65 to 75° C., usually from about 67 to 73° C. and maintained for a period of time ranging from about 15 sec to 20 min, usually from about 30 sec to 5 min.

The above cycles of denaturation, annealing, and polymerization may be performed using an automated device, typically known as a thermal cycler. Thermal cyclers that may be employed are described elsewhere herein as well as in U.S. Pat. Nos. 5,612,473; 5,602,756; 5,538,871; and 5,475,610; the disclosures of which are herein incorporated by reference.

The methods described herein can also be used in non-PCR based applications to detect a target nucleic acid sequence, where such target may be immobilized on a solid support. Methods of immobilizing a nucleic acid sequence on a solid support are known in the art and are described in Ausubel et ah, eds. (1995) Current Protocols in Molecular Biology (Greene Publishing and Wiley-Interscience, NY), and in protocols provided by the manufacturers, e.g., for membranes: Pall Corporation, Schleicher &amp; Schuell; for magnetic beads: Dynal; for culture plates: Costar, Nalgenunc; for bead array platforms: Luminex and Becton Dickinson; and, for other supports useful according to the embodiments provided herein, CPG, Inc.

Variations on the exact amounts of the various reagents and on the conditions for the PCR or other suitable amplification procedure (e.g., buffer conditions, cycling times, etc.) that lead to similar amplification or detection/quantification results are known to those of skill in the art and are considered to be equivalents. In one embodiment, the subject QPCR detection has a sensitivity of detecting fewer than 50 copies (preferably fewer than 25 copies, more preferably fewer than 15 copies, still more preferably fewer than 10 copies, e.g. 5, 4, 3, 2, or 1 copy) of target nucleic acid (i.e., KPC nucleic acids) in a sample. In one embodiment, a hot-start PCR reaction is performed (e.g., using a hot start Taq DNA polymerase) so as to improve PCR reaction by decreasing background from non-specific amplification and to increase amplification of the desired extension product. The methods disclosed herein advantageously enable the user to detect clinically relevant levels of carbapenem-resistant pathogens in samples. For example, the methods disclosed herein can, in preferred embodiments, detect less than 10⁹ CFU/ml, preferably less than 10⁸ CFU/ml, more preferably less than 10⁷, 10⁶, 10⁵, 10⁴, and less than 10³ CFU/ml.

### Controls

The assays disclosed herein can optionally include controls. PCR or QPCR reaction of the present invention may contain various controls. Such controls can include a “no template” negative control, in which primers, buffer, enzyme(s) and other necessary reagents (e.g., MgCl₂, nucleotides, and the like) are cycled in the absence of added test sample. This ensures that the reagents are not contaminated with polynucleotides that are reactive with the primers, and that produce spurious amplification products. In addition to “no template” controls, negative controls can also include amplification reactions with non-specific target nucleic acid included in the reaction, or can be samples prepared using any or all steps of the sample preparation (from nucleic acid extraction to amplification preparation) without the addition of a test sample (e.g., each step uses either no test sample or a sample known to be free of carbapenem-resistant microorganisms).

In some embodiments, the methods disclosed herein can include a positive control, e.g., to ensure that the methods and reagents are performing as expected. The positive control can include known target that is unrelated to the bla_{KPC} target nucleic acids disclosed herein. Prior to amplification, the positive control nucleic acid (e.g., in the form of a plasmid that is either linearized or non-linearized) can be added to the amplification reaction. A single reaction may contain either a positive control template, a negative control, or a sample template, or a single reaction may contain both a sample template and a positive control. Preferably, the positive control will comprise sequences that are substantially complementary to the bla_{KPC} forward and reverse amplification primers derived from the blame sequences disclosed herein, such that an amplification primer pair used to amplify bla_{KPC} sequences will also amplify control nucleic acids under the same assay conditions. In some embodiments, the amplicon generated from the positive control template nucleic acids is larger than the target amplicon. Preferably, aside from the sequences in a positive control nucleic acid that are complementary or substantially complementary to the forward and reverse primers, the positive control nucleic acid will not share substantial similarity with the target amplicon/bla_{KPC} sequences disclosed herein. In other words, outside from the forward and reverse primers, the positive control amplicon is preferably less than 80%, less than 70%, less than 60%, less than 50%, less that 40%, less than 30%, less than 20%, and even more preferably, less than 10% identical with the positive control polynucleotide, e.g., when the sequence identity is compared using NCBI BLAST ALIGN tools.

For example, in some embodiments, the methods disclosed herein include providing a positive control that consists of, consists essentially of, or comprises SEQ ID NO:18, or a variant thereof, to which the forward and reverse blaKPC primers SEQ ID NO: 1 and 2 are completely complementary. The blaKPC probe of SEQ ID NO:3, by contrast, shares no significant homology to, and will not specifically hybridize to SEQ ID NO:18. In some embodiments, a positive control probe can be provided, that will specifically hybridize to positive control nucleic acid sequences, e.g., positive control amplicons. By way of example, in some embodiments, the compositions and methods disclosed herein include a positive control probe that is substantially identical to SEQ ID NO:19, that will specifically hybridize to positive control amplicon sequences amplified from SEQ ID NO: 18.

Positive and negative controls can be used in setting the parameters within which a test sample will be classified as having or not having a bla_{KPC} gene, responsible for conferring carbapenem resistance.

For example, in a QPCR reaction, the cycle threshold at which an amplicon is detected in a positive control sample can be used to set the threshold for classifying a sample as “positive,” and the cycle threshold at which an amplicon is detected in a negative control sample can be used to set the threshold for classifying a sample as “negative.” The CT from a single reaction may be used for each control, or the median or mean of replicate samples may be used. In yet another embodiment, historical control values may be used. The minimum level of detection for each of the negative and the positive controls is typically set at the lower end of the 95% confidence interval of the mean CT across multiple reactions. This value can be adjusted depending on the requirements of the diagnostic assay.

Preferably, PCR controls should be performed at the same time as the test sample, using the same reagents, in the same amplification reaction.

Some embodiments provide for the determination of the identity and/or amount of target amplification products, during the amplification reaction, e.g., in real-time. For example, some embodiments relate to taking measurements of, for example, probe that is specifically bound to target amplicon nucleic acids, and/or positive control amplicons (e.g., as indicated by fluorescence). Measurements may be taken at a specified point during each cycle of an amplification reaction, e.g., after each extension step (prior to each denaturation step). In alternative embodiments, measurements of the amount of probe that is specifically bound to target amplicon nucleic acids, and/or positive control amplicons can be taken continuously throughout each cycle.

Alternatively, in some embodiments, the identity/amount of the amplicons (e.g., target and/or positive control) can be confirmed after the amplification reaction is completed, using standard molecular techniques including (for example) Southern blotting, dot blotting and the like.

### Kits

Also provided herein are kits containing the reagents and compositions to carry out the methods described herein. Such a kit can comprise a carrier being compartmentalized to receive in close confinement therein one or more containers, such as tubes or vials. One of the containers may contain at least one unlabeled or detectably labeled primer or probe disclosed herein. The primer or primers can be present in lyophilized form or in an appropriate buffer as necessary. One or more containers may contain one or more enzymes or reagents to be utilized in PCR reactions. These enzymes may be present by themselves or in admixtures, in lyophilized form or in appropriate buffers.

Finally, the kit can include all of the additional elements necessary to carry out the methods disclosed herein, such as buffers, extraction reagents, enzymes, pipettes, plates, nucleic acids, nucleoside triphosphates, filter paper, gel materials, transfer materials, autoradiography supplies, and the like.

The kits according to the present invention will comprise at least: (a) a labeled oligonucleotide, where the kit includes two or more distinguishable oligonucleotides, e.g., that hybridize to a nucleotide sequence encoding a carbapenemase gene; and (b) instructions for using the provided labeled oligonucleotide(s) in a high fidelity amplification, e.g., PCR, reaction, such as QPCR. In one embodiment the two distinguishable oligonucleotides will be selected from the group consisting of SEQ ID NOS: 1-17 and 19.

In some embodiments, the kits include additional reagents that are required for or convenient and/or desirable to include in the reaction mixture prepared during the methods disclosed herein, where such reagents include: one or more polymerases; an aqueous buffer medium (either prepared or present in its constituent components, where one or more of the components may be premixed or all of the components may be separate), and the like. The various reagent components of the kits may be present in separate containers, or may all be pre-combined into a reagent mixture for combination with template nucleic acid.

In addition to the above components, in some embodiments, the kits can also include instructions for practicing the methods disclosed herein. These instructions can be present in the kits in a variety of forms, one or more of which may be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address that may be used via the internet to access the information at a removed site. Any convenient means may be present in the kits.

### Examples

The following examples are provided to demonstrate particular situations and settings in which this technology may be applied and are not intended to restrict the scope of the invention and the claims included in this disclosure.

The first example demonstrates that the compositions and methods disclosed herein are useful and extremely sensitive tools for the detection and identification of the presence of pathogens harboring carbapenemase genes of any of the known isoforms. As demonstrated below, the compositions and methods disclosed herein advantageously detect carbapenemase nucleic acids from various bacterial species, in various sample matrices.

Initial Amplification of KPC Genes from Cell Lysates and Isolated Genomic DNA

Crude cell lysates and purified genomic DNA samples were extracted from twenty (20) bacterial strains listed below, including isolates of Klebsiella pneumoniae, Enterobacter cloacae, Pseudomonas aeruginosa, Enterobacter aerogenes and Klebsiella oxytoca that were previously determined to be bla_{KPC} positive or bla_{KPC} negative. The strains used are listed in Table 2.

| | | | | crude |
|---|---|---|---|---|
| internal | | KPC | | DNA |
| ID | Species | genotype | gDNA | (lysate) |
| IDI-3592 | Klebsiella pneumoniae | KPC-1 | x | x |
| IDI-3991 | Klebsiella pneumoniae | KPC-1 | | x |
| IDI-3593 | Klebsiella pneumoniae | KPC-2 | x | x |
| IDI-3733 | Enterobacter cloacae | KPC-2 * | | x |
| IDI-3992 | Klebsiella pneumoniae | KPC-2 | | x |
| IDI-3993 | Pseudomonas aeruginosa | KPC-2 | | x |
| IDI-4403 | Klebsiella pneumoniae | KPC-2 | | x |
| IDI-3475 | Escherichia coli | KPC-3 | x | x |
| IDI-4405 | Klebsiella pneumoniae | KPC-3 | | x |
| IDI-4057 | Escherichia coli | KPC-4 | x | x |
| IDI-4056 | Pseudomonas aeruginosa | KPC-5 | x | x |
| IDI-4059 | Escherichia coli | KPC-5 | | x |
| IDI-3441 | Enterobacter aerogenes | unknown * | | x |
| IDI-3468 | Escherichia coli | unknown | | x |
| IDI-3533 | Klebsiella oxytoca | unknown | | x |
| IDI-3581 | Klebsiella pneumoniae | unknown | | x |
| IDI-285 | Escherichia coli | No bla_{KPC} | | x |
| IDI-2020 | Enterococcus faecalis | No bla_{KPC} | | x |
| IDI-3552 | Klebsiella pneumoniae | No bla_{KPC} | | x |
| IDI-3790 | Pseudomonas aeruginosa | No bla_{KPC} | x | x |

* These strains were identified as KPC but negative results were obtained using the primers and assay described in Yigit et al., 2001.

### Preparation of Purified Genomic DNA Samples:

Isolated colonies from fresh cultures were suspended in 10 mL of TSB and incubated for 23 h at 37° C. From those bacterial suspensions, a pre-lysis step was performed as follow: the bacterial suspensions were centrifuged 10 min at 3860 rpm, the pellets were suspended with 1 mL of PBS and centrifuged 5 min at 3860 rpm.

For other species than E. coli: Cell pellets were suspended with 100 μL of PBS. The suspensions were transferred into lysis tubes (BD Diagnostics, Québec, Canada) and vortexed at high speed for 10 min. After a quick spin step, 120 μL of PBS was added into each tube and lysates were incubated at 95° C. for 2 min. A volume of 190 μL of the lysate was mixed to 10 μL of RNAse, vortexed, centrifuged and heated 10 min at 37° C.

For E. coli: Cell pellets were suspended with 150 μL of PBS. The suspensions were heated 2 min at 95° C. 10 μL of RNAse was added into each tube. The tubes were vortexed and centrifuged quickly. Lysates were heated 5 min at 37° C. 20 μL of proteinase K was added to each tube. The tubes were gain vortexed and centrifuged quickly, followed by heating for 30 min at 55° C.

DNA extraction was performed on MAGTRATION® nucleic acid extraction instrument (PSS Bio Instruments, Pleasanton, Calif.) according to the manufacturer's instructions. The quantity and the quality of the purified genomic DNAs were analyzed using a spectrophotometer and agarose gel electrophoresis.

### Preparation of Crude Sample Lysates for Direct Analysis:

Isolated colonies from fresh cultures were suspended in TE (1×) to an OD that was equivalent to a McFarland 0.5 standard corresponding to ˜1×10⁵ copies/μL. 50 μL of cell suspensions were transferred to lysis tubes, vortexed at high speed for 5 min and incubated at 95° C. for 2 min. The lysates were stored at ˜20° C. for later use.

### Preparation of a QPCR Master Mix:

DNA and/or lysates prepared as described above were tested in QPCR reactions at the following concentrations: 10,000 copies/μL, 100 copies/μL, 30 copies/μL and ˜21 copies/μL.

In addition, samples were spiked with an internal control (“IC”) nucleic acid at different concentrations. The internal control nucleic acid used in the assay is a vector that includes sequences that are complementary to both the forward and reverse KPC amplification primers used in the assay, and an unrelated sequence located between the binding sites for the KPC forward and reverse primers, such that the unrelated sequence is amplifiable with the primers under standard PCR conditions. The expected size of the internal control amplicon generated using the IC nucleic acid is longer that the expected size of the KPC target amplicon.

The master mix was prepared to provide QPCR ready samples with the following components: lx Fast Start PCR buffer (Roche, Mannheim, Germany); 2-5 mM MgCl₂, 015 mM dNTPs, 0.4 μM KPC forward primer; 0.4 μM KPC reverse primer; 0.35 μM KPC molecular beacon; 0.2-0.6 μM IC molecular beacon; 3-36 copies IC DNA/μL; 0.15 mg/mL BSA; 0-4% trehalose. FastStart Taq polymerase was added to a final concentration of 0.09 units.

Amplification reactions were performed in a Rotor-Gene™ 6000 instrument (Corbett Life Sciences). The samples were cycled as follows:

| | | Temp | | |
|---|---|---|---|---|
| Stage | Status | (° C.) | Sec | Optics |
| 1 | Hold | 95 | 900 | off |
| 2 | Repeat 45 | 95 | 1-5 | off |
| | times | 56-58 | 9, 10, or 15 | on |
| | | 72 | 10-20 | off |

Detection of KPC gene target was monitored in the FAM channel whereas the internal control was monitored in the TET channel.

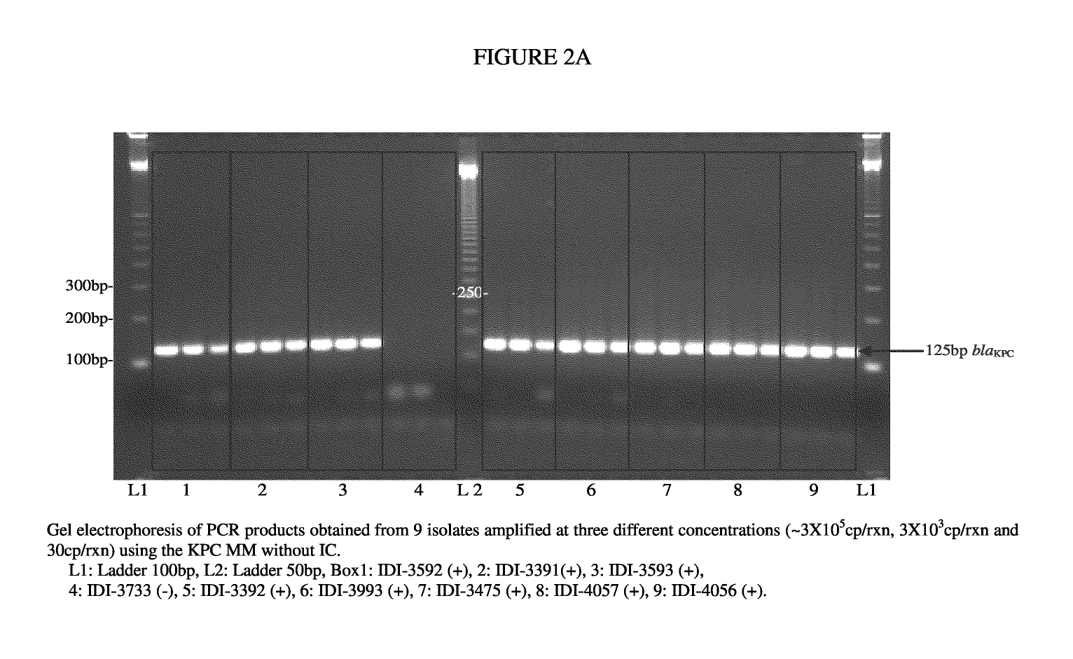

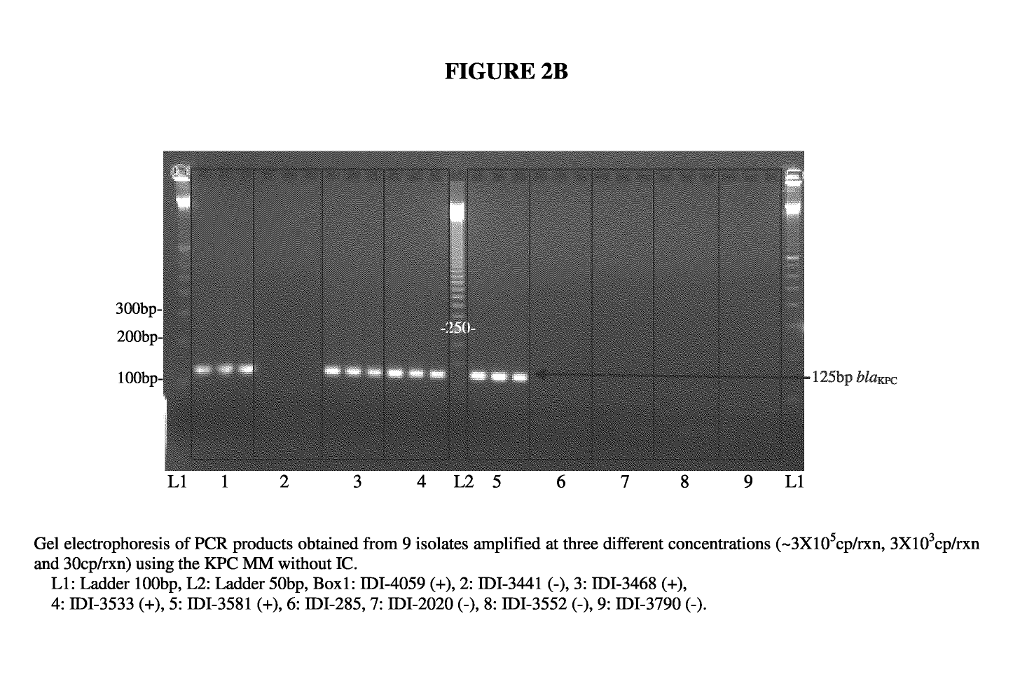

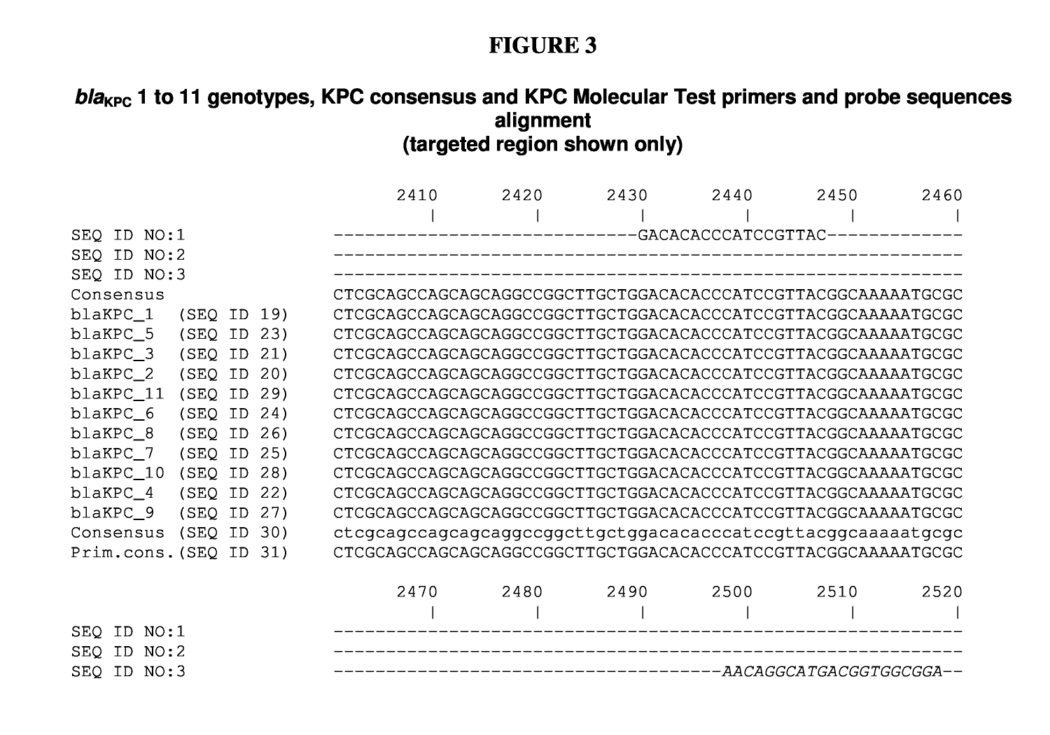

A portion of each of the amplified samples was analyzed via gel electrophoresis following the amplification protocol listed above. The results are shown in FIGS. 2A and 2B. The results demonstrate that the methods described herein can detect as little as 30 copies of template DNA per reaction. The inclusion of a positive control template and a positive control probe did not adversely affect the amplification of the bla_{KPC} target sequences.

PCR Amplification from Rectal, Wound, and Urine Specimens

The sample preparation of rectal and wound specimens was performed using the BD GeneOhm™ Lysis Kit, according to the manufacturer's protocol provided in sections A-1, A-2 (i, ii, iii and v) and E. Swabs used in the preparation of the samples were BBL™ CultureSwab™ Liquid Stuart (Becton Dickinson, Franklin Lakes, N.J.) and BBL™ CultureSwab™Liquid Amies single or double swabs (Becton Dickinson, Franklin Lakes, N.J.). The sample preparation of urine specimens was performed using the BD GeneOhm™ Lysis Kit, according to the manufacturer's protocol in sections B (concentration method), D (washing method) and E (lysis method). The real concentration of the diluted suspensions was determined by colony count (IT-040-032). Briefly, 50 μL of the 3 lowest suspensions (n=3 for each) were spread on BAP and incubated over night at 35° C. These dilutions were selected to obtain 30 to 300 CFU per plate. After the growth period, counts and averages of the replicates per concentration were performed.

To determine the limit of detection (“LOD”) using rectal and wound samples, a series of 30 swabs were soaked in a volume of 75 μL of the bacterial suspensions at 10 different concentrations. For LOD in urine matrix, 75 μL of bacterial suspensions at 10 different concentrations were added to 1 mL of negative urine matrix. After bacterial suspensions were properly absorbed by the swabs (in case of LOD with rectal and wound specimens), sample preparation was performed as described above. The results were as follows:

| Species | Matrix | CFU/rxn | CFU/swab |
|---|---|---|---|
| Klebsiella pneumoniae | rectal | 1 | 404 |
| Escherichia coli | rectal | 0.8 | 283 |
| Klebsiella pneumoniae | wound | 0.8 | 263 |
| Klebsiella pneumoniae | urine | 4 | 66 CFU/mL |

The data indicate that the methods disclosed herein provide an extremely high analytical sensitivity with all matrices and bacterial species tested. The presence of internal control nucleic acids and an internal control probe did not adversely affect the sensitivity of the reactions.

The invention described and claimed herein is not to be limited in scope by the specific embodiments herein disclosed, since these embodiments are intended as illustrations of several aspects of the invention. Any equivalent embodiments are intended within the scope of this invention. Indeed, various modifications of the embodiments in addition to those shown and described herein will become apparent to those skilled in the art from the foregoing description. The appended claims are intended to cover such modifications.

What is claimed is:

1. A kit for the detection of isoforms 1-11 of KPC beta-lactamases (bla_{KPC1-11}), comprising:
a forward amplification primer; and
a reverse amplification primer, wherein said forward and reverse amplification primers are substantially complementary to SEQ ID NOs: 19-29, or the complement thereof, throughout the entire length of the primers, and wherein said forward and reverse amplification primers are together capable of specifically amplifying a target amplicon from SEQ ID NOs: 19-29.

2. The kit of claim 1, further comprising a probe, wherein said probe comprises a nucleic acid sequence that is substantially complementary to at least a portion of the target amplicon.

3. The kit of claim 1, wherein said oligonucleotide probe comprises a detectable moiety on its 3′ end.

4. The kit of claim 1, wherein said oligonucleotide probe comprises a detectable moiety on its 5′ end.

5. The kit of claim 1, wherein said forward and reverse amplification primers are lyophilized.

6. The kit of claim 2, wherein said probe is lyophilized.

7. The kit of claim 1, further comprising deoxynucleotides.

8. The kit of claim 1, further comprising a PCR buffer.

9. The kit of claim 1, further comprising a positive control nucleic acid, wherein said positive control nucleic acid comprises a sequence substantially complementary to the forward primer and a sequence that is substantially complementary to the reverse primer, and wherein the remainder of the positive control nucleic acid is not substantially complementary to any one of SEQ ID NOs: 19-29, or the complements thereof.

10. The kit of claim 1, wherein said forward primer and said reverse primer each comprise between 10 to 45 nucleotides, wherein said forward primer comprises at least 10 consecutive nucleotides of SEQ ID NO:1, and wherein said reverse primer comprises at least 10 consecutive nucleotides of SEQ ID NO:2.

11. The kit of claim 1, wherein said forward primer consists of SEQ ID NO:1, or a variant thereof, wherein said variant can include 1 to 5 nucleotide additions or deletions at its 5′ end, its 3′ end or both, and 1 to 5 degenerate bases,
wherein said reverse primer consists of SEQ ID NO:2, or a variant thereof, wherein said variant can include 1 to 5 nucleotide additions or deletions at its 5′ end, its 3′ end or both, and 1 to 5 degenerate bases.

12. The kit of claim 2, wherein said probe comprises an oligonucleotide between 10 and 45 bases in length, and wherein at least 15 consecutive bases of the oligonucleotide are substantially complementary a sequence within the target amplicon.

13. The kit of claim 11, wherein said probe comprises an oligonucleotide between 10 and 45 bases in length, and wherein said oligonucleotide comprises SEQ ID NO:3.

14. The kit of claim 11, wherein said probe comprises an oligonucleotide, wherein said oligonucleotide consists of SEQ ID NO:15.

15. A method for determining the presence of a carbapenem-resistant pathogen in a sample, comprising:
providing the sample;
contacting the sample with a forward amplification primer and a reverse amplification primer, wherein said forward and reverse amplification primers are substantially complementary to SEQ ID NOs: 19-29, or the complement thereof, throughout the entire length of the primer, and wherein said forward and reverse amplification primers are together capable of specifically amplifying a target amplicon from SEQ ID NOs: 19-29, wherein said contacting occurs under standard PCR conditions and wherein the target amplicon is generated provided that the sample comprises the carbapenem-resistant pathogen to generate an amplified sample; and
determining whether the target amplicon is present in the amplified sample.

16. The method of claim 15, wherein the determining step comprises contacting the amplified sample with a probe, wherein the probe comprises a detectable moiety, and wherein said detectable moiety generates a signal in the presence of the target amplicon.

17. The method of claim 16, wherein said probe comprises an oligonucleotide sequence that is substantially complementary to at least a portion of the target amplicon.

18. The method of claim 15, wherein said generation of the amplified sample comprises real time PCR.

19. The method of claim 15, wherein said forward primer consists of SEQ ID NO:1, or a variant thereof, wherein said variant can include 1 to 5 nucleotide additions or deletions at its 5′ end, its 3′ end or both, and 1 to 5 degenerate bases,
wherein said reverse primer consists of SEQ ID NO:2, or a variant thereof, wherein said variant can include 1 to 5 nucleotide additions or deletions at its 5′ end, its 3′ end or both, and 1 to 5 degenerate bases.

20. The method of claim 16, wherein said probe comprises an oligonucleotide between 10 and 45 bases in length, and wherein at least 15 consecutive bases of the oligonucleotide are substantially complementary a sequence within the target amplicon.

21. The method of claim 16, wherein said probe comprises an oligonucleotide between 10 and 45 bases in length, and wherein said oligonucleotide comprises SEQ ID NO:3.

22. The method of claim 21, wherein said probe comprises an oligonucleotide, wherein said oligonucleotide consists of SEQ ID NO:15.

23. The method of claim 15, further comprising:
providing a positive internal control nucleic acid, wherein said positive control nucleic acid comprises a sequence substantially complementary to the forward primer and a sequence that is substantially complementary to the reverse primer, and wherein the remainder of the positive control nucleic acid is not substantially complementary to any one of SEQ ID NOs: 19-28, or the complements thereof; and
contacting said positive control with said forward and said reverse amplification primer under said standard PCR conditions to generate a positive control amplicon.

24. A method for determining the presence of sequences of nucleic acids encoding isoforms 1-11 of KPC beta-lactamases (bla_{KPC1-11}), or fragments of the nucleic acids encoding isoforms 1-11 of KPC beta-lactamases (bla_{KPC-11}):
providing a sample to be tested for the presence of KPC beta-lactamases (bla_{KPC-11});
contacting the sample with a forward amplification primer and a reverse amplification primer, wherein said forward and reverse amplification primers are substantially complementary to SEQ ID NOs: 19-29, or the complement thereof throughout the entire length of the amplification primers, and wherein said forward and reverse amplification primers are together capable of specifically amplifying a target amplicon from SEQ ID NOs: 19-29, wherein said contacting occurs under standard PCR conditions and wherein the target amplicon is generated provided that the sample comprises the carbapenem-resistant pathogen to generate an amplified sample; and
determining whether the target amplicon is present in the amplified sample.


SEQUENCE LISTING (32 sequences)

SEQ ID NO: 1 - DNA, 18 bp, Artificial Sequence
  Synthetic oligoncuelotide
gacacacccatccgttac

SEQ ID NO: 2 - DNA, 19 bp, Artificial Sequence
  Synthetic oligoncuelotide
ggcgttatcactgtattgc

SEQ ID NO: 3 - DNA, 21 bp, Artificial Sequence
  Synthetic oligoncuelotide
aacaggcatgacggtggcgga

SEQ ID NO: 4 - DNA, 18 bp, Artificial Sequence
  Synthetic oligoncuelotide
aactgacactgggctctg

SEQ ID NO: 5 - DNA, 18 bp, Artificial Sequence
  Synthetic oligoncuelotide
atacmctccgcaggttcc

SEQ ID NO: 6 - DNA, 21 bp, Artificial Sequence
  Synthetic oligoncuelotide
acacgaccggcaaccaccgca

SEQ ID NO: 7 - DNA, 18 bp, Artificial Sequence
  Synthetic oligoncuelotide
gatrgataccggctcagg

SEQ ID NO: 8 - DNA, 18 bp, Artificial Sequence
  Synthetic oligoncuelotide
gtaacggatgggtgtgtc

SEQ ID NO: 9 - DNA, 20 bp, Artificial Sequence
  Synthetic oligoncuelotide
gctgccgctgtgctggctcg

SEQ ID NO: 10 - DNA, 18 bp, Artificial Sequence
  Synthetic oligoncuelotide
cggcttgctggacacacc

SEQ ID NO: 11 - DNA, 18 bp, Artificial Sequence
  Synthetic oligoncuelotide
caggccggcttgctggac

SEQ ID NO: 12 - DNA, 18 bp, Artificial Sequence
  Synthetic oligoncuelotide
tacggcaaaaatgcgctg

SEQ ID NO: 13 - DNA, 19 bp, Artificial Sequence
  Synthetic oligoncuelotide
caacaaattggcggcggcg

SEQ ID NO: 14 - DNA, 19 bp, Artificial Sequence
  Synthetic oligoncuelotide
tatcactgtattgcacggc

SEQ ID NO: 15 - DNA, 33 bp, Artificial Sequence
  Synthetic oligoncuelotide
gcagcgaacaggcatgacggtggcggacgctgc

SEQ ID NO: 16 - DNA, 35 bp, Artificial Sequence
  Synthetic oligoncuelotide
cgcgatcacacgaccggcaaccaccgcagatcgcg

SEQ ID NO: 17 - DNA, 34 bp, Artificial Sequence
  Synthetic oligoncuelotide
cgcgatcgctgccgctgtgctggctcggatcgcg

SEQ ID NO: 18 - DNA, 4244 bp, Artificial Sequence
  positive control vector
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttgacacacccatccgttacgaaggtcggttacaaacagtcaccggagtagagatgttgaaattgcaggcaaattgattgatttcaccagctgtattagaagtacaagaaggttggttacaacccaaagacagctgtgcatgaattgcagaaaatttattgcagcttcgccacaggaaaggtggagcaatgtgaagaggcatcatgccatctgctgtaggctatcaaccaatggtaagactcttctggaagcaattgagctatggtcatgccaggtgacaacatatgatgagtcatgcaatacagtgataacgccaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 19 - DNA, 180 bp, Artificial Sequence
  blaKPC_1
ctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagt

SEQ ID NO: 20 - DNA, 180 bp, Artificial Sequence; identical to SEQ ID NO: 19
  blaKPC_2

SEQ ID NO: 21 - DNA, 180 bp, Artificial Sequence; identical to SEQ ID NO: 19
  blaKPC_3

SEQ ID NO: 22 - DNA, 180 bp, Artificial Sequence
  blaKPC_4
ctcgcagccagcagcaggccggcttgctggacacacccatccgttacggcaaaaatgcgctggttcggtggtcacccatctcggaaaaatatctgacaacaggcatgacggtggcggagctgtccgcggccgccgtgcaatacagtgataacgccgccgccaatttgttgctgaaggagt

SEQ ID NO: 23 - DNA, 180 bp, Artificial Sequence; identical to SEQ ID NO: 22
  blaKPC_5

SEQ ID NO: 24 - DNA, 180 bp, Artificial Sequence; identical to SEQ ID NO: 19
  blaKPC_6

SEQ ID NO: 25 - DNA, 180 bp, Artificial Sequence; identical to SEQ ID NO: 19
  blaKPC_7

SEQ ID NO: 26 - DNA, 180 bp, Artificial Sequence; identical to SEQ ID NO: 19
  blaKPC_8

SEQ ID NO: 27 - DNA, 180 bp, Artificial Sequence; identical to SEQ ID NO: 19
  blaKPC_9

SEQ ID NO: 28 - DNA, 180 bp, Artificial Sequence; identical to SEQ ID NO: 22
  blaKPC_10

SEQ ID NO: 29 - DNA, 180 bp, Artifiical Sequence
  blaKPC_11
ctcgcagccagcagcaggccggcttgctggacacacccatccgttacggcaaaaatgcgctggttctgtggtcacccatctcggaaaaatatctgacaacaggcatgacggtggcggagctgtccgcggccgccgtgcaatacagtgataacgccgccgccaatttgttgctgaaggagt

SEQ ID NO: 30 - DNA, 180 bp, Artificial Sequence; identical to SEQ ID NO: 19
  blaKPC_consensus

SEQ ID NO: 31 - DNA, 180 bp, Artificial Sequence; identical to SEQ ID NO: 19
  blaKPC_consensus

SEQ ID NO: 32 - DNA, 40 bp, Artificial Sequence
  synthetic oligonucleotide
cggcgatgcctcttcacattgctccacctttcctcgccgg